(19) United States
(12) Patent Application Publication
(10) Pub. No.: US 2017/0252199 A1
(43) Pub. Date: Sep. 7, 2017

(54) INNER-SIDE PILLAR SUPPORTER

(71) Applicant: Hiroji FUKUTA, Kasugai-shi (JP)

(72) Inventor: Hiroji FUKUTA, Kasugai-shi (JP)

(21) Appl. No.: 15/509,623

(22) PCT Filed: Aug. 28, 2015
(86) PCT No.: PCT/JP2015/004363

§ 371 Date: Mar. 8, 2017

(30) Foreign Application Priority Data
Sep. 10, 2014 (JP) 2014-184664

(51) Int. Cl. A61F 5/058

(52) U.S. Cl. CPC A61F 5/0585; A61F 5/05825

(57) ABSTRACT

An inner-side pillar supporter according to the present invention includes a plate-shaped pillar including: a fixed portion coming into contact with an inner side of an ankle joint at the malleolus medialis from an inner side of a calcaneus, and an inversion preventing portion formed integrally with the fixed portion to extend over the malleolus medialis; and a fixing member press fixing the fixed portion of the pillar onto the inner side of the ankle joint at the malleolus medialis from the inner side of the calcaneus.

### TECHNICAL FIELD

The present invention relates to an inner-side pillar supporter for restricting an ankle joint from inverting.

### BACKGROUND ART

When an ankle is twisted inward, an anterior talofibular ligament, tibiofibular ligament or posterior talofibular ligament, each of which is an outer-side ligament of the ankle, is extended or damaged to cause ankle-joint inversion sprains. In an initial therapy for the ankle-joint inversion sprains, a so-called “RICE” treatment has been carried out. In the “RICE” treatment, an affected area is made unmovable to keep it at rest. Then, the affected area is cooled to make bloodstreams gentle, preventing the affected area from swelling or breeding internally. Moreover, the affected area is compressed moderately to control swellings and inflammations. In addition, a patient is laid on his or her back to place the affected area at a position that is present higher than is the heart present. Thus, bloodstreams are kept from flowing into the affected area, inhibiting inflammations.

In a later therapy, a supporter for an ankle joint has been used in most cases. The supporter restricts the inversions of an ankle, which occur due to the extensions or damages of outer-side ligaments, in order to fix the ankle joint at the ordinary position.

As an example of the supporter for an ankle joint, a supporter set forth in Japanese Unexamined Utility Model Publication (KOKAI) Gazette No. 3-101930 will be explained below. The supporter for an ankle joint comprises a supporter body to be mounted or applied to the upper side and lower side of an ankle joint involving the ankle of a foot, a stay attached to an outer side of the supporter body in order to prevent twists, and a belt to be would around for fixation.

### RELATED TECHNICAL LITERATURE

### Patent Literature

Patent Literature No. 1: Japanese Unexamined Utility Model Publication (KOKAI) Gazette No. 3-101930

### SUMMARY OF THE INVENTION

### Assignment to be Solved by the Invention

The conventional supporter for an ankle joint has been heretofore restricting the ankle joint from inverting, and simultaneously has been tending to restrict a foot's plantar/dorsal flexing movements as well.

Hence, it is an assignment to the present invention to provide an ankle-joint supporter not only restricting an ankle joint from inverting but also being free of restricting the plantar/dorsal flexing movements at the ankle joint.

### Means for Solving the Assignment

An inner-side pillar supporter according to the present invention comprises:

a plate-shaped pillar including a fixed portion coming into contact with an inner side of the ankle joint at a malleolus medialis thereof from an inner side of a calcaneus, and an inversion preventing portion formed integrally with the fixed portion to extend over the malleolus medialis; and

a fixing member press fixing the fixed portion of the pillar from the inner side of the calcaneus onto the inner side of the ankle joint at the malleolus medialis.

The inner-side pillar supporter according to the present invention comprises the pillar whose fixed portion is fixed by the fixing member to a lower section of a foot's ankle to be integrated with a lower section of the ankle joint. The inversion preventing portion of the pillar is located above off from the lower section of the ankle joint so that it is located on an inner side in an upper section of the ankle in the ankle joint. Accordingly, the inversion preventing portion of the pillar prevents the ankle joint from inverting during which the upper section of the ankle joint inclines toward the inner side. Meanwhile, an upper section of the ankle joint is capable of swinging in the front/rear direction by way of the ankle joint' lower section and the ankle. The fixing portion neither fixes the upper section of the ankle joint, nor the inversion preventing portion of the pillar. Consequently, the ankle joint is capable of swinging at the upper section in the front/rear direction, without ever being restrained by the inversion preventing portion of the pillar, namely, it is capable of carrying out planar/dorsal flexing movements.

The inner-side pillar supporter according to the present invention comprises the plate-shaped pillar, and the fixing member fixing the pillar to the ankle joint at the downside. The pillar has a plated shape, and includes: the fixed portion coming into contact with an inner side in the lower section of the ankle joint (namely, an inner side of the ankle joint at the malleolus medialis) from an inner side of a calcaneus to be fixed to the inner side in the lower section of the ankle joint; and the inversion preventing portion formed integrally with the fixed portion to extend over the ankle joint (namely, over the malleolus medialis). The plate-shaped pillar can be the same pillar (or stay) as that of the conventional supporter for an ankle joint. If such is the case, a lower section of the conventional pillar makes the fixed portion according to the present invention, and its upper section makes the inversion preventing portion according to the present invention.

Since the fixed portion of the pillar is fixed at the lower section to an inner side in the lower section of the ankle joint, the fixed portion can preferably have a configuration following or going along with an inner-side configuration of the ankle joint, and can preferably exhibit a sufficient coming-into-contact area. The inversion preventing portion at the upper section of the pillar not only needs to have a sufficient height making it possible to prevent inversions, but also needs to have such a sufficient horizontal width that the ankle joint does not stick at the upside from out of the inversion preventing portion even for the ankle joint that is swinging at the upside.

The fixing member according to the present invention fixes the fixed portion of the pillar to the lower section of the ankle joint. Such a fixing member as fixing the ankle joint at the upper section as well is unemployable. Employable as the fixing member according to the present invention is members or parts that fix the fixed portion of the pillar to the lower section of the ankle joint by the fixation to the lower section of the ankle joint alone. Note that the fixing member and the pillar can preferably be integrated one another at the time of use at least. Fixing the fixed portion of the pillar to the lower section of the ankle joint results in fixing the fixed portion more securely to the lower section of the ankle joint on the inner side.

Specifically, it is possible to adopt, as the fixing member according to the present invention, a bag-shaped, a socks-shaped, or a belt-shaped fixing means fixing the ankle joint only at the lower section. As the belt-shaped fixing member, a strip-shaped bandage is preferable. The strip-shaped bandage can preferably be fixed at one of the opposite ends to the pillar detachably or integrally therewith. Moreover, the strip-shaped bandage can preferably comprise: a strip-shaped body portion wound around the ankle joint to fix the ankle joint; and a fastening means fastening the wound-around body portion at a trailing end thereof onto a face side of the body portion, the face side disposed on a more leading-end side of the body portion than is the trailing end. In addition, the body portion can preferably include: a central strip portion having a slit that extends in a longitudinal direction thereof, and into which a heel fits; a leading-end strip portion extending from the central strip portion to a leading-end side thereof, and having a sufficient length to circle around an ankle once at least to be connected to the central strip portion into which the heel has been fitted; and a trailing-end strip portion extending from the central strip portion to a trailing-end side thereof, and having a length that extends from the central strip portion, into which the heel has been fitted, to circle around the ankle once at least. Fitting an ankle into the slit in the strip-shaped bandage makes the bandage fix the ankle joint more securely, making the fixation of the pillar onto the ankle-joint lower section more reliable.

### Effect of the Invention

The inner-side pillar supporter according to the present invention comprises the pillar whose fixed portion is fixed to a lower section in a foot's ankle by the fixing member so that the fixed portion is integrated with a lower section of the ankle joint. The inversion inhibiting portion of the pillar is located up above off from the lower section of the ankle joint so that the inversion inhibiting portion is located on an inner side in the upper section of the ankle in the ankle joint. Accordingly, the inversion inhibiting portion of the pillar inhibits the ankle joint from inverting in which the ankle joint inclines inward at the upper section. Meanwhile, the ankle joint is swingable at the upper section in the forward/rear direction by way of the ankle joint's lower section and the ankle. The fixing member neither fixes the upper section of the ankle joint nor the inversion inhibiting portion of the pillar. Consequently, the ankle joint is capable of swinging at the upper section in the front/rear direction, namely, it is capable of performing plantar/dorsal flexing movements at the ankle joint, without ever being restrained by the inversion inhibiting portion of the pillar. Moreover, the inversion inhibiting portion of the pillar can inhibit the ankle joint from inverting even at any place during the plantar/dorsal flexing movements at the ankle joint, because the inversion inhibiting portion has a horizontal width that is wide sufficiently.

### BRIEF DESCRIPTION OF THE DRAWINGS

FIG. 1 illustrates a front view, side view, “A”-“A” cross-sectional view and “B”-“B” cross-sectional view of an inner-side pillar supporter according to Embodiment;

FIG. 2 illustrates a plan view of a strip-shaped bandage of the inner-side pillar supporter according to Embodiment;

FIG. 3 illustrates a plan view of a fastening means of the strip-shaped bandage shown in FIG. 2; and

FIG. 4 illustrates how to mount or apply the inner-side pillar supporter.

### MODE FOR CARRYING OUT THE INVENTION

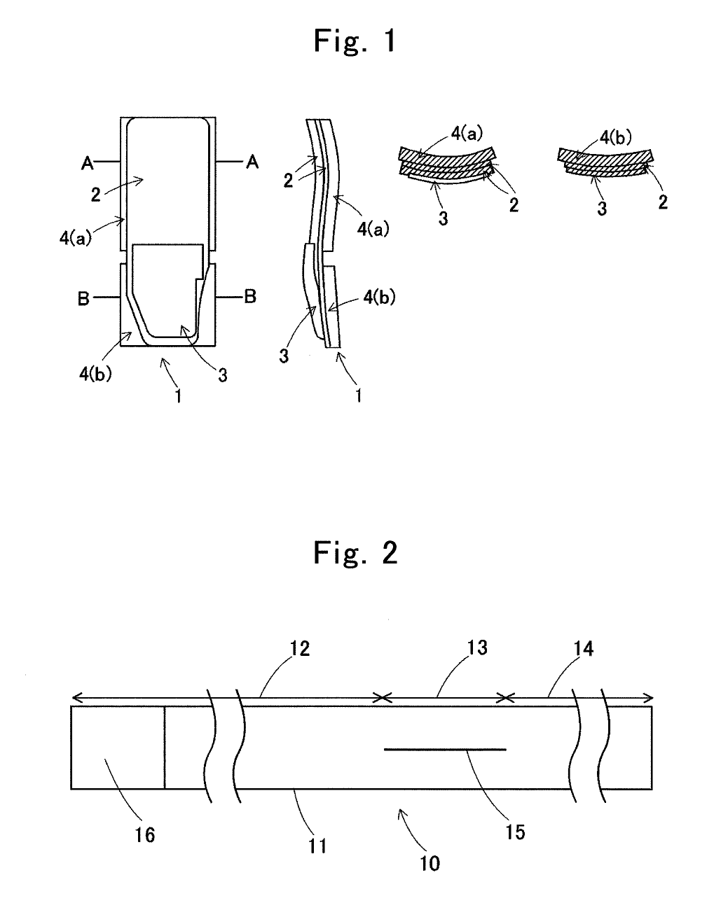

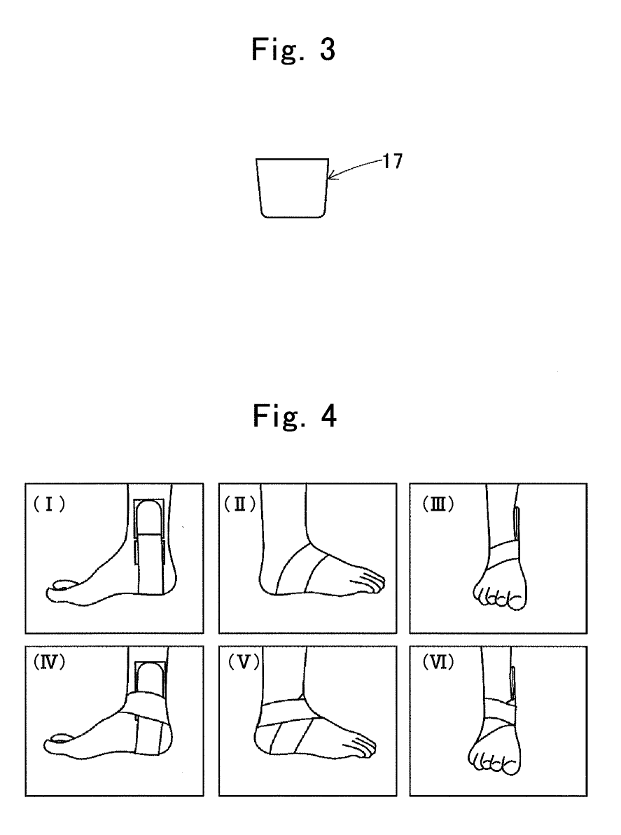

FIG. 1 through FIG. 3 illustrate an inner-side pillar supporter according to Embodiment of the present invention. The inner-side pillar supporter comprises a pillar 1 shown in FIG. 1, a strip-shaped bandage 10 shown in FIG. 2, and a planar fastener 17 shown in FIG. 3.

The pillar 1 includes a bent plate 2 made of plastic, an upside cushioning material 4(a) and downside cushioning material 4(b) made of sponge, and a male planar fastener 3 formed as a hook shape. As can be understood from the plan view shown in FIG. 1, the bent plate 2 makes a configuration that has a rectangular shape with a broad width at the upside, and whose width narrows down toward the lower end at the downside. As can be understood from the side view therein, the bent plate 2 makes an inverted letter-“S” configuration that has an upper-end section warping in a direction obliquely to the left on the diagram, and a lower-end section waring slightly in another direction obliquely to the right thereon. In addition, as can be understood from the cross-sectional views therein, the bent plate 2 makes a bent plate whose cross section curves as an arc shape. Sizes of the bent plate 2 are 15.2 cm in the longitudinal direction, 5.2 cm at the upside in the width direction, and 2 mm in the thickness direction.

On a section involving an upside on the inner side of the bent plate 2, the upside cushioning material 4(a) is fixed; whereas the downside cushioning material 4(b) is fixed on the lower section while the latter is separated off from the former by a minor clearance provided therebetween. The planar fastener 3 is fixed to the bent plate 2 at the lower side on the outer side. The upside cushioning material 4(a) has 8.3 cm in the longitudinal direction, 5.2 cm in the width direction, and a thickness of 5 mm. The downside cushioning material 4(b) has 6 cm in the longitudinal direction, 5.2 cm in the width direction, and a thickness of 5 mm. The planar fastener 3 has 7 cm in the longitudinal direction, a maximum length of 4.7 cm in the width direction, and a minimum length of 2 cm in the width direction.

The upside cushioning material 4(a) comes into contact with an upper section in an ankle at an ankle joint, and is then fixed to the upper section; whereas the downside cushioning material 4(b) comes into contact with the ankle's lower section, and is then fixed to the lower section. Thus, the ankle comes to be located between the upside cushioning material 4(a) and the downside cushioning material 4(b).

The bent plate 2 is constructed as described above. That is, the downside cushioning material 4(b), the section in the bent plate 2 retaining the downside cushioning material 4(b), and the section involving the planar fastener 3 constitute the fixed portion according to the present invention; whereas the upside cushioning material 4(a), and the section in the bent plate 2 retaining the upside cushioning material 4(a) constitute the inversion preventing portion according to the present invention.

The strip-shaped bandage 10 includes a strip-shaped body portion 11 shown in FIG. 2, and a planar fastener 17 shown in FIG. 3.

The strip-shaped body portion 11 has 80.5 cm in the longitudinal direction, 5.1 cm in the width direction, and a thickness of 1 mm. The strip-shaped body portion 11 is provided with a female planar fastener having a large number of fine rings on the surface over the entire face. Note that the female planar fastener has 80.5 cm in the longitudinal direction, 4.4 cm in the width direction, and a thickness of 1 mm.

The strip-shaped body portion 11 is formed of such three strip portions as a leading-end strip portion 12, a central strip portion 13, and a trailing-end strip portion 14. The leading-end strip portion 12 has 56.2 cm in the longitudinal direction, 5.1 cm in the width direction, and a thickness of 1 mm. The leading-end strip portion 12 is provided with a planar fastener 16 at the free end. Note that the planer fastener 16 has 7.3 cm in the longitudinal direction, 4.4 cm in the width direction, and a thickness of 1 mm.

The central strip portion 13 has 9.3 cm in the longitudinal direction, 5.1 cm in the width direction, and a thickness of 1 mm. The central strip portion 13 is provided with a slit 15 into which an ankle is fitted at the time of mounting or applying. The slit 15 is a 9.3-cm cut or opening that is parallel to the longitudinal direction of the central strip portion 13, and is arranged at the middle in the width direction of the central strip portion 13.

The trailing-end strip portion 14 has 15 cm in the longitudinal direction, 5.1 cm in the width direction, and a thickness of 1 mm.

The planar fastener 17 has a male planar fastener on the surface, and is formed roughly as a trapezoidal configuration whose lower base is 4.8 cm, upper base is 3 cm and height is 4 cm.

The inner-side pillar supporter according to Embodiment comprises the above-mentioned constituents.

Next, an example of mounting or applying the inner-side pillar supporter will be hereinafter described using FIG. 4. Before mounting or applying the inner-side pillar supporter to an ankle joint, the planar fastener 16, which is fixed to the leading end of the strip-shaped bandage 10, is pressed and then applied to the planar fastener 3, which is fixed to the outer side in the bent plate 2 of the pillar 1 at the lower side, to put the strip-shaped bandage 10 in a state where it extends straight from the lower side of the pillar 1 as shown at (I) in FIG. 4. Thereafter, as shown at (I) in FIG. 4, the strip-shaped body portion 11 is elongated downward as follows: the upside cushioning material 4(a) is brought into contact with an upper side in the ankle so as to make the ankle in the ankle joint meet or face the space that the pillar 1 includes between the upside cushioning material 4(a) and the downside cushioning material 4(b); and then the downside cushioning material 4(b) is brought into contact with an inner side of the ankle joint so as to locate it below the ankle. Next, as shown at (II), the leading-end strip portion 12 is circled around to the outer side of the foot by winding it around the sole that intervenes from the plantar arch to the heel. Next, as shown at (III), the leading-end strip portion 12 is passed around from the ankle (i.e., the outer side of the foot to the front of the leg's slender part above the foot). Next, as shown at (IV), the leading-end strip portion 12 is circled around from the front of the ankle to the rear of the ankle while fixing the fixed portion of the pillar 1. Next, as shown at (V), the leading-end strip portion 12 is circled around from the rear of the ankle to the front of the ankle through the outer side of the foot. Next, as shown at (VI), the leading-end strip portion 12 is circled around from the front of the ankle while fixing the fixed portion at the lower side. Thereafter, the leading-end strip portion 12 is passed around the foot to the outer side after hooking the heel onto the slit 15 formed in the central strip portion 13 and then fitting the heel into the slit 15. Moreover, the trailing-end strip portion 14 is circled around the fixed portion from the front of the ankle. Finally, the planar fastener 17 is used on the front side of the strip-shaped body portion 11 to fasten the inner-side pillar supporter according to Embodiment whose body portion 11 has been put in a circled-around state.

The inversion prohibiting portion, which exists from the upper side to the middle section in the pillar 1 to restrict the ankle from inverting, is not fixed at all. Therefore, the inversion prohibiting portion does not restrict plantar/dorsal flexing movements at the ankle joint, because it can swing freely in the front/rear direction of the foot depending on the plantar/dorsal flexing movements. Moreover, it is easy for a patient to perform the plantar/dorsal flexing movements, because the downside cushioning material 4(b) and the ankle joint slide one another.

As a result, the inner-side pillar supporter according to Embodiment not only restricts the ankle joint from inverting but also is fully free of restricting the plantar/dorsal flexing movements at the ankle joint.

(Employment Evaluation)

The inner-side pillar supporter according to present Embodiment was used to treat 24 cases of fresh ankle-joint inversion sprain for patients who visited an orthopedic clinic operated by the applicant of the present application. For 10 cases of minor injury, the inner-side pillar supporter was mounted or applied to the patients from the beginning. For the other 14 cases, the inner-side pillar supporter was mounted or applied to the patients after they had undergone a cast fixation for from five days to three weeks approximately. In all of the cases, the inner-side pillar supporter was always mounted or applied to the patients other than such occasions as the patients took off the inner-side pillar supporter from the ankle joint to take a bath or shower, and the like. Then, at the time when swellings around the ankle-joint malleolus lateralis and oppressive pains thereat had disappeared, the patients were instructed to mount or apply the inner-side pillar supporter to them only during their activities.

At a time three months later after being affected by the injury, it was possible to return the patients back to their original activity levels for all of the 10 minor-injury cases, and for 12 cases among the other 14 cases. No satisfactory results like these have been obtained by conventionally-available supporters.

### EXPLANATION ON REFERENCE NUMERALS

1: Pillar;

2: Bent Plate;

3: Pillar Planar Fastener;

4(a): Upside Cushioning Material;

4(b): Downside Cushioning Material;

10: Strip-shaped Bandage;

11: Strip-shaped Body Portion;

12: Leading-end Strip Portion;

13: Central Strip Portion;

14: Trailing-end Strip Portion;

15: Slit;

16: Leading-end Planar Fastener; and

17: Planar Fastener

1.-7. (canceled)

8. An inner-side pillar supporter comprising:
a plate-shaped pillar including a fixed portion coming into contact with an inner side in a downside of an ankle joint at a malleolus medialis thereof from an inner side of a calcaneus, and an inversion preventing portion formed integrally with the fixed portion to extend over an upside of the ankle joint at the malleolus medialis; and
a fixing member press fixing the fixed portion of the pillar from the inner side of the calcaneus onto the inner side in the downside of the ankle joint at the malleolus medialis in such a state as neither the upside of the ankle joint nor the inversion preventing portion of the pillar is fixed.

9. The inner-side pillar supporter as set forth in claim 8, wherein the inversion preventing portion of the pillar has such a sufficient horizontal width that the upside of the ankle joint does not stick from out of the inversion preventing portion even when the ankle joint swings at the upside in the front/rear direction.

10. The inner-side pillar supporter as set forth in claim 8, wherein the fixing member includes a strip-shaped bandage.

11. The inner-side pillar supporter as set forth in claim 10, wherein the pillar and one of the opposite ends of the bandage are fixed one another.

12. The inner-side pillar supporter as set forth in claim 10, wherein the bandage comprises: a strip-shaped body portion wound around the ankle joint to fix the ankle joint; and a fastening means fastening the wound-around body portion at a trailing end thereof onto a face side of the body portion, the face side disposed on a more leading-end side of the body portion than is the trailing end;
the body portion including:
a central strip portion having a slit that extends in a longitudinal direction thereof, and into which a heel fits;
a leading-end strip portion extending from the central strip portion to a leading-end side thereof, and having a sufficient length to circle around an ankle once at least to be connected to the central strip portion into which the heel has been fitted; and
a trailing-end strip portion extending from the central strip portion to a trailing-end side thereof, and having a length that extends from the central strip portion, into which the heel has been fitted, to circle around the ankle once at least.

13. The inner-side pillar supporter as set forth in claim 10 further comprising a detachable fixing means at a section thereof where the fixed portion comes into contact with the bandage.

14. The inner-side pillar supporter as set forth in claim 8, wherein the fixed portion includes a cushioning material on a face thereof that is disposed face-to-face to the inner side of the ankle joint, the cushioning material coming into contact with the inner side of the ankle joint.
